(19) United States
(12) Patent Application Publication
(10) Pub. No.: US 2024/0248087 A1
(43) Pub. Date: Jul. 25, 2024

(54) PROTEOMIC SAMPLE INVESTIGATION METHOD FOR DIAGNOSING RHABDOMYOLYSIS AND KITS FOR IMPLEMENTING SAME

(71) Applicants: CENTRO DE EDUCAÇÃO FÍSICA ALMIRANTE ADALBERTO NUNES, Rio de Janeiro (BR); UNIVERSIDADE FEDERAL DO RIO DE JANEIRO, Rio de Janeiro (BR)

(72) Inventors: Marcos DIAS PEREIRA, Rio de Janeiro (BR); Andréia CARNEIRO DA SILVA, Rio de Janeiro (BR); Giuseppe PALMISANO, São Paulo (BR)

(73) Assignees: CENTRO DE EDUCAÇÃO FÍSICA ALMIRANTE ADALBERTO NUNES, Rio de Janeiro, RJ (BR); UNIVERSIDADE FEDERAL DO RIO DE JANEIRO, Rio de Janeiro, RJ (BR)

(21) Appl. No.: 18/564,216

(22) PCT Filed: Sep. 23, 2022
(86) PCT No.: PCT/BR2022/050375

§ 371 Date: Nov. 27, 2023

(30) Foreign Application Priority Data
Oct. 14, 2021 (BR) 1020210206136

(51) Int. Cl. G01N 33/573; G01N 33/68

(52) U.S. Cl. CPC G01N 33/573; G01N 33/6848; G01N 33/6893; G01N 2333/7055; G01N 2333/78; G01N 2333/902; G01N 2333/976; G01N 2333/99; G01N 2570/00; G01N 2800/10

(57) ABSTRACT

Methods and kits used in the diagnosis of predisposition to the development of rhabdomyolysis. To perform the invention, a biological sample is withdrawn from an investigated individual, such as a blood or urine sample, preferably urine. The investigation is conducted from the detection and quantification of proteins in said sample, proteins selected from a panel that includes: cathepsin H; alpha-1 collagen chain (I); phosphatidylinositol-3-kinase interaction protein 1; beta-defensin 1; integrin beta-1; Brevican core protein; a member of the tumor necrosis factor receptor superfamily 10C; gamma-glutamylcyclotransferase; Glutaredoxin, Flavin reductase; Desmocollin-2; Alpha-1 collagen chain (I); Sodium/nucleoside cotransporter 1; Uteroglobin; Hemoglobin alpha subunit; Hemicentin-1; CCN family member 3; Parkinson's disease protein 7; Intercellular adhesion molecule 2; Secreted Ly-6/uPAR-related protein 1; N-acetylmuramoyl-L-alanine amidase; Cathepsin Z; Fatty acid-binding protein 5; Atractin; Peptidyl-prolyl cis-trans isomerase A and heart fatty acid-binding protein.

### CROSS REFERENCE TO RELATED APPLICATIONS

This application is the US National Phase of and claims the benefit of and priority on International Application No. PCT/BR2022/050375 having an international filing date of 23 Sep. 2022, which claims priority on and the benefit of Brazilian Patent Application No. 10 2021 020613 6 having a filing date of 14 Oct. 2021.

### BACKGROUND OF THE INVENTION

### Technical Field

The present description belongs to the field of investigation or analysis of materials by specifics methods, directed at biological material, involving proteins, peptides or amino acids.

### Prior Art

In the medical field, investigation and diagnosis of diseases is a crucial stage in choosing the appropriate treatment and mitigating the pathology. Consequently, using non-invasive, sensitive and effective biochemical analysis methods become excellent identification tools.

Exertional rhabdomyolysis (ER) can be observed in high-performance athletes and military personnel subjected to intense repetitive and prolonged exercises. ER is characterized by involving damage to striated muscles with release of cellular components into the extracellular fluid and bloodstream, causing imbalance to the organism's homeostasis.

Due to cellular extravasation, protein, electrolytes and enzymes show elevated levels in the bloodstream. Consequently, it is possible to investigate biomarkers involved in the physiopathology of the disease. In clinical practice, the investigation of blood levels of creatine kinase (CK), detection of myoglobinuria and assessment of renal function are used for the diagnosis of rhabdomyolysis.

In order to identify and quantify the progression of rhabdomyolysis under a current view, the proteomic, metabolomic and genomic study emerges as strategies in the investigation of specific biomarkers, in the assessment of the extension of tissue damage caused and, also, in the detection of genetic predispositions.

Mohan's and collaborators' work (2019, Pharmacognosy Magazine, 15, S359-S365) evaluated the potential muscle intoxication caused by Chata-edulis, which is a plant capable to induce to rhabdomyolysis. The detection of the protein HFABP in rat blood samples was associated with cardiac damage resulting from induced rhabdomyolysis. The utilization of this HFABP protein as a marker in human blood or urine is not anticipated.

Sampson and collaborator (2013, Expert review of proteomics, 11, 91-106.) investigated the literature in order to evidence the proteomic and metabolomic of urine, correlating it to performance of physical activity at different degrees of intensity. Among the various substances mentioned, the HFABP protein and fragments of collagen type 1 stood out, however without offering interpretation related to rhabdomyolysis.

Balfoussia and collaborators (2013, Journal of Proteomics, 98, 1-14) reported a proteomic study in athletes' plasma, in order to evaluate the metabolic changes caused by extreme physical stress. Thirty proteins were pointed as indicative of inflammation. It was concluded that physical stress alters the composition of circulating proteins in the organism and that, in turn, they can act as biomarkers of diseases related to high physical effort load. The target proteins of the study are not included in the scope of the present patent application.

Pereira's and collaborators' publication (2019, Arquivos Brasileiros de Cardiologia, 113, 294-298.) reported a case of Rhabdomyolysis after military training in a male individual. For the evaluative methodology of plasma alterations, biomarkers traditionally associated with the development of Rhabdomyolysis were explored, being these CK and interleukins (IL). The association of these markers with clinical conditions of microvascular dysfunction and Rhabdomyolysis was discussed.

A proteomic study to identify markers of muscular dystrophy was carried out by Gargan and collaborators (2020, Molecular omics, 16, 268-278). The investigation was conducted in urine of mice model of dystrophy and compared to their proteomic profile (profile available on the Open Science Framework—OSF, em https://osf.io/). There is some similarity between muscular dystrophies and rhabdomyolysis due to the clinical condition triggered and the presence of some markers. However, they are diseases that have different causes and biochemical profiles.

Bacha and collaborators (2016, Dissertação Mestrado em Ciências Farmacêuticas, UFAM) studied genetic polymorphisms and biochemical changes associated with Rhabdomyolysis in military personnel. Changes in serum levels of CK and electrolytes above the baseline limits confirmed the presence of polymorphins in genes of interest, indicating that they could influence the damage caused to the muscle tissue.

Quintas and collaborators (2020, Metabolomics, 16, art. 45) analyzed the metabolomic of urine from professional soccer players. The work showed the relationship between the changes in the metabolic profile and the development of muscle injuries.

Document EP2761289B1 discloses a method of detecting biomarkers in biological samples (which can be a blood, urine, serum or plasma sample), and assessing the risk of a Cardiovascular Event (CV). Cathepsin H is displayed in the panel of makers proposed in the prior art as predictive of Cardiovascular Event. The disclosed method is not related to the diagnosis of Rhabdomyolysis or musculoskeletal damage resulting from physical exercise.”

The prior art points out several documents that can be related to the diagnosis of Rhabdomyolysis, clinical picture of muscle injuries, as well as the biomarkers involved in the investigations of biological material. That being said, it is noted that the prior art does not disclose a method that is capable of predicting the early diagnosis of ER, comprising at least two biomarkers from the panel of biomarkers presented in the present application, being these: cathepsin H (CTSH); collagen alpha-1 (I) chain (CO1A1); phosphatidylinositol-3-kinase interaction protein 1 (PIK3IP1); beta-defensin 1 (DEFB1); integrin beta-1 (ITGB1); Brevican core protein (BCAN); tumor necrosis factor receptor superfamily member 10C (TNFRSF10C); gamma-glutamylcyclotransferase (GGCT); Glutaredoxin (GLRX1); Flavin reductase (NADPH) (BLVRB); Desmocollin-2 (DSC2); Sodium/nucleoside cotransporter 1 (S28A1); Uteroglobin (UTER); Hemoglobin alpha subunit (HBA); Hemicentin-1 (HMCN1); CCN family member 3 (CCN3); Parkinson's disease protein 7 (PARK7); Intercellular adhesion molecule 2 (ICAM2); Secreted Ly-6/uPAR related protein 1 (SLURP1); N-acetylmuramoyl-L-alanine amidase (PGRP2); Cathepsin Z (CATZ); Fatty acid binding protein 5 (FABP5); Atractin (ATRN); Peptidyl-prolyl cis-trans isomerase A (PPIA) and heart fatty acid binding protein (FABP3).

### BRIEF SUMMARY OF THE INVENTION

It is one of the objectives of the present description to disclose a proteomic investigation method for assessing the disposition to develop ER, a clinical syndrome that lacks both specific diagnostic methods and protein markers that evidence the anticipation and progression of the disease and that allow interventions prior to the critical stages of the disease.

The objectives of the present description are achieved by the disclosure of a protein biomarker panel, comprising: cathepsin H (CTSH); collagen alpha-1 (I) chain (CO1A1); phosphatidylinositol-3-kinase interaction protein 1 (PIK3IP1); beta-defensin 1 (DEFB1); integrin beta-1 (ITGB1); Brevican core protein (BCAN); a member of the tumor necrosis factor receptor superfamily 10C (TNFRSF10C); gamma-glutamylcyclotransferase (GGCT); Glutaredoxin (GLRX), Flavin reductase (NADPH) (BLVRB); Desmocollin-2 (DSC2); Collagen alpha-1 (I) chain (COL1A1); Sodium/nucleoside cotransporter 1 (S28A1); Uteroglobin (UTER); Hemoglobin alpha subunit (HBA); Hemicentin-1 (HMCN1); CCN family member 3 (CCN3); Parkinson's disease protein 7 (PARK7); Intercellular adhesion molecule 2 (ICAM2); Secreted Ly-6/uPAR related protein 1 (SLURP1); N-acetylmuramoyl-L-alanine amidase (PGRP2); Cathepsin Z (CATZ); Fatty acid binding protein 5 (FABP5); Atractin (ATRN); Peptidyl-prolyl cis-trans isomerase A (PPIA) and heart fatty acid binding protein (FABPH or FABP3); wherein, given a biological sample withdrawn from an individual, the detection of significant differences in the relative abundance of at least two of the biomarkers of said sample in relation to a set of reference values (e.g., p-value<0.05 in relation to reference sample values) indicates the disposition for development of ER.

### BRIEF DESCRIPTION OF THE DRAWINGS

The present invention is found illustrated in the embodiments represented in figures, as briefly described below.

FIG. 1 is a schematic flowchart of an embodiment of the present method, for monitoring the progression of ER risk from the analysis of a biological sample.

FIGS. 2A to 2H, 3A to 3H and 4A to 4I are a series of graphs with the receiver operating characteristic (ROC) curves for evaluating the performance of the proposed binary classification system for the biomarkers identified, according to the method of the present description, in biological samples of a group of individuals before and after being subjected to strenuous physical activities.

FIG. 5 is a histogram with results of a proteomic assay in biological samples of a group of individuals before and after being subjected to strenuous physical activities, graphically identifying proteins differentially regulated, overexpressed and underexpressed.

FIG. 6 shows the analysis of the principal components (PCA) of the total proteins identified and quantified in biological samples of a group of individuals before and after being subjected to strenuous physical activities.

FIGS. 7A to 7D are a series of graphs that quantify the levels of the classical markers of rhabdomyolysis in the prior art (LDH, AST, CKMB and CK) in biological samples of a group of individuals before and after being subjected to strenuous physical activities.

FIG. 8 is a Volcano Plot scatter plot showing differentially regulated proteins in biological samples of a group of individuals before and after being subjected to strenuous physical activities.

FIGS. 9 and 10 are graphs of the evolution of the urinary proteome of a patient hospitalized due to RE, after undergoing military training, presenting data from the pre-training collection, the hospitalization period and the time of hospital discharge.

### DETAILED DESCRIPTION OF PREFERRED EMBODIMENTS

The object herein disclosed is a proteomic investigation method in a sample to diagnose disposition to develop rhabdomyolysis in individuals subjected to strenuous physical exercises, such as those practiced in a military environment, in high-performance sports modalities training and in competition conditions. The method consists of detecting significant variations in the relative abundance of a panel of biomarkers in said sample.

For the scope of the present description, a panel of biomarkers is understood as a set of at least two biomarkers. A diagnostic method that investigates at least two biomarkers will use a panel of markers.

According to the method of the present invention, it is disclosed a panel of biomarkers comprising: cathepsin H (CTSH); collagen alpha-1 (I) chain (CO1A1); phosphatidylinositol-3-kinase interaction protein 1 (PIK3IP1); beta-defensin 1 (DEFB1); integrin beta-1 (ITGB1); Brevican core protein (BCAN); a member of the tumor necrosis factor receptor superfamily 10C (TNFRSF10C); gamma-glutamylcyclotransferase (GGCT); Glutaredoxin (GLRX1); Flavin reductase (NADPH) (BLVRB); Desmocollin-2 (DSC2); Sodium/nucleoside cotransporter 1 (S28A1); Uteroglobin (UTER); Hemoglobin alpha subunit (HBA); Hemicentin-1 (HMCN1); CCN family member 3 (CCN3); Parkinson's disease protein 7 (PARK7); Intercellular adhesion molecule 2 (ICAM2); Secreted Ly-6/uPAR related protein 1 (SLURP1); N-acetylmuramoyl-L-alanine amidase (PGRP2); Cathepsin Z (CATZ); Fatty acid binding protein 5 (FABP5); Atractin (ATRN); Peptidyl-prolyl cis-trans isomerase A (PPIA) and heart fatty acid binding protein (FABP3).

In one embodiment, the present invention contemplates a signature expression profile comprising a panel of urinary protein biomarkers, wherein said profile is used to diagnose ER. In one embodiment, the panel of biomarkers comprises a plurality of overexpressed urinary proteins. In one embodiment, the panel of biomarkers comprises a plurality of underexpressed urinary proteins. In one embodiment, the plurality of overexpressed urinary proteins is selected from the group consisting of CTSH, GGCT, PGRP2, HBA, PARK7, CATZ, FABP5 and PPIA. In one embodiment, the plurality of underexpressed urinary proteins is selected from the group consisting of PIK3IP1, DEFB1, ITGB1, BCAN, TNFRSF10C, GLRX1, NADPH, BLVRB, DSC2, CO1A1, S28A1, UTER, HMCN1, CCN3, ICAM2, SLURP1 and ATRN.

In one embodiment of the present method for assessing the risk of progression to rhabdomyolysis in an individual, it is necessary that at least two markers of said panel, regardless of the number of evaluated markers, present significant variations in relative abundance when compared to reference values. Therefore, in an evaluation that investigates two, three, four, five, six, or more proteins of the described panel of markers, at least two significant changes must be presented for it to be considered a health condition of risk for progression or development of ER.

The terms “strenuous physical activity”, “intense physical exercise”, “strenuous physical training” and their obvious variants are understood as any physical activities classified, either through technical definitions or individual perceptions of the performer of the activity, as those that cause physical and respiratory discomfort, due to strenuous motor activities.

In accordance with the previous definition, the physical activities considered in this document refer to high-performance physical training, whether they are sports or military, participation in sports and/or military competitions, including all modalities of marathons, physical training based on very intense exercises, such as CrossFit and similar ones.

Due to the sometimes individual and subjective character of this interpretation, activities such as climbing stairs, jumping, playing recreationally, bathing/swimming recreationally are also considered, as they may configure intense or excessive exercise within the claimed scope.

Physical activities practiced in adverse climatic conditions, such as in high or low temperatures, within inadequate hydroelectrolytic replacement and long periods, also cause effects similar to those of strenuous physical activity, being therefore included in this definition.

The concept of appropriate diagnostic thresholds can be approached in various ways, depending on the study in question. For example, a recommended diagnostic threshold for the diagnosis of acute myocardial infarction using cardiac troponin is the 97.5 percentile of the concentration observed in a normal population. Another method may be to look at serial samples from the same patient, where a previous “baseline” result is used to monitor temporal changes in a biomarker level.

Population studies can also be used to select a decision limit. The use of receiver operating characteristic (ROC) curves arose from the field of signal detection theory developed during World War II for the analysis of radar images, and ROC analysis are often used to select a threshold capable of better distinguishing a “ill” subpopulation from a “subpopulation without disease”. A false positive, in this case, occurs when the person's test is positive, but actually does not have the disease. A false negative, on the other hand, occurs when the person tests negative, suggesting that they are healthy, when in fact they have the disease. To draw a ROC curve, the true positive rate (TPR) and the false positive rate (FPR) are determined as the decision limit is continuously varied. Since TPR is equivalent to sensitivity and FPR is equal to 1-specificity, the ROC graph is sometimes called the sensitivity vs (1-specificity) graph. A perfect test will have an area under the ROC curve of 1.0; a random test will have an area of 0.5. A limit is selected to provide an acceptable level of specificity and sensitivity.

In this context, “ill” refers to a population with a characteristic (the presence of a disease or condition or the occurrence of some outcome) and “not ill” is intended to refer to a population without the characteristic. Although a single decision limit is the simplest application of such a method, multiple decision limits can be used. For example, below a first threshold, the absence of disease can be attributed with relatively high confidence and, above a second threshold, the presence of disease can also be attributed with relatively high confidence. Between the two limits it can be considered indeterminate. This was done to be exemplary only in nature.

In addition to threshold comparisons, other methods for correlating assay results to a patient classification (occurrence or non-occurrence of disease, probability of an outcome, etc.) include decision trees, sets of rules, Bayesian methods and neural network methods. These methods can produce probability values that represent the degree wherein a subject belongs to a classification of a plurality of classifications.

Test accuracy measures can be obtained as described in Fischer et al., Intensive Care Med. 29: 1043-51, 2003, and used to determine the efficacy of a particular biomarker. These measures include sensitivity and specificity, predictive values, likelihood ratios, diagnostic odds ratios, and areas of the ROC curve. The area under the curve (“AUC”) of a ROC graph is equal to the probability that a classifier classifies a randomly chosen positive instance higher than a randomly chosen negative one. The area under the ROC curve can be considered equivalent to the Mann-Whitney U test, which tests the median difference between the scores obtained in the two groups considered if the groups are of continuous data, or to the Wilcoxon signed-rank test.

As discussed above, suitable tests may exhibit one or more of the following results in these various measures: a specificity greater than 0.5, preferably at least 0.6, more preferably at least 0.7, even more preferably at least 0.8, even more preferably at least 0.9 and most preferably at least 0.95, with a corresponding sensitivity greater than 0.2, preferably greater than 0.3, more preferably greater than 0.4, even more preferably at least 0.5, even more preferably 0.6, even more preferably greater than 0.7, even more preferably greater than 0.8, more preferably greater than 0.9, and more preferably greater than 0.95; a sensitivity greater than 0.5, preferably at least 0.6, more preferably at least 0.7, even more preferably at least 0.8, even more preferably at least 0.9 and most preferably at least 0.95, with a corresponding specificity greater than 0.2, preferably greater than 0.3, more preferably greater than 0.4, even more preferably at least 0.5, even more preferably 0.6, even more preferably greater than 0.7, even more preferably greater than 0.8, more preferably greater than 0.9, and more preferably greater than 0.95; sensitivity of at least 75%, combined with specificity of at least 75%; an area of ROC curve greater than 0.5, preferably at least 0.6, more preferably 0.7, even more preferably at least 0.8, even more preferably at least 0.9, and most preferably at least 0.95; a probability ratio different from 1, preferably at least about 2 or more or about 0.5 or less, more preferably at least about 3 or more or about 0.33 or less, even more preferably at least about 4 or more or about 0.25 or less, even more preferably at least about 5 or more or about 0.2 or less, and most preferably at least about 10 or more or about 0.1 or less; a positive likelihood ratio (calculated as sensitivity/(1-specificity)) greater than 1, at least 2, more preferably at least 3, even more preferably at least 5, and most preferably at least 10; and/or a negative likelihood ratio (calculated as (1-sensitivity)/specificity) less than 1, less than or equal to 0.5, more preferably less than or equal to 0.3, and most preferably less than or equal to 0.1.

Although some markers presented in the panel of the present method, when singly considered, have known association with cardiovascular events or genetic conditions related to muscle conduction, the biomarker panel described herein, and also the grouping into subsets of at least two biomarkers is specifically indicated as a marker panel for prediction of ER.

The present method, in addition to providing prognostic risk of development of ER based on the measurements of proteins that constitute the said panel, also allows a mapping of the effects of different degrees of physical exercise on the health of individual evaluated during physical activity practice, allowing the creation of physical training programs that prevent the development of RE, as well as personalized physical training programs. Therefore, the present diagnostic method contributes to the development of training strategies that contemplate the prevention of ER and its serious outcomes such as permanent impairment of renal functions or death.

In one embodiment of the method, a biological sample is incubated with an antibody or a specific protein fragment that binds specifically to a protein or peptide fragment of a protein of the said diagnostic panel, under conditions that allow the antibody or specific protein fragment to form a complex with (a) a peptide fragment of a protein of the said panel; or (b) the covalent or non-covalent complex of at least one molecule selected from the group comprising: peptide fragments of a protein of the said panel; and, intact proteins of the said panel. It is followed by the step of detecting and measuring the complex formed. According to this embodiment of the diagnostic method, the presence and level of modification products of the proteins of the panel in a biological sample are detected and quantified by means of known methods, whether colorimetric or fluorimetric or chemiluminescent methods, for example, by a kit/assay of enzymatic immunoadsorption type ELISA.

The prior art comprises a variety of mass spectrometer configurations, which can be used to detect biomarker values. In general, a mass spectrometer has the following main components: a sample input, an ion source, a mass analyzer, a detector, a vacuum system and an instrument control system and a data system. The difference in the sample input, ion source and mass analyzer usually defines the type of instrument and its capabilities. For example, an input can be a liquid chromatography source of capillary column or can be a direct or indirect probe, such as used in matrix-assisted laser desorption ionization. Common ion sources are, for example, electrospray, including nanospray and microspray or matrix-assisted laser desorption ionization. Common mass analyzers include a quadrupole mass filter, ion trap mass analyzer and time of flight mass analyzer.

In one embodiment, protein biomarkers and biomarker values can be detected and measured by: Mass Spectrometry with Electrospray Ionization (ESI-MS), ESI-MS/MS, ESI-MS/(MS)n, Mass Spectrometry by Matrix-Assisted Laser Desorption Ionization and Time of Flight Analyzer (MALDI-TOF-MS), Surface Enhanced Laser Desorption/Ionization Mass Spectrometry and Time of Flight Analyzer (SELDI-TOF-MS), Silicon Desorption/Ionization (DIOS), Secondary lon Mass Spectrometry (SIMS), Quadrupole-Time of Flight (Q-TOF), Tandem Time of Flight (TOF/TOF), Ultraflex III TOF/TOF technology, Mass Spectrometry by Atmospheric Pressure Chemical Ionization (APCI-MS), APCI-MS/MS, APCI-(MS)n, Mass Spectrometry by Atmospheric Pressure Photoionization (APPI-MS), APPI-MS/MS and APPI-(MS)n, Fourier Transform Mass Spectrometry (FTMS), Quantitative Mass Spectrometry, and Mass Spectrometry with Ion Trap Analyzer.

In one embodiment, any combination of the disclosed biomarkers can be detected using a suitable kit, for use in performing the method disclosed herein. In addition, any kit may contain two or more detectable markers, for example, by fluorescence emission, chemiluminescence, turbidimetry and nephelometry.

In one embodiment, a kit includes one or more capture reagents (such as, for example, an antibody) for detecting two or more biomarkers in a biological sample, wherein the biomarkers include any two of the biomarkers disclosed in the panel of this document, and optionally (b) one or more software for classifying the individual from whom the biological sample was obtained as having or not risk of progression to a rhabdomyolysis condition, as described herein. One or more instructions for manually performing the above steps by a human may be provided as an alternative to the software or computer programs.

A kit means a detection device that combines a solid support with a corresponding capture reagent having a signal generating material. The kit may also include instructions for use of the systems and reagents, sample handling and data analysis. In addition, the kit can be used with a computer system or software to analyze and report the result of the analysis of the biological sample. The kits may also contain one or more reagents (for example, solubilization buffers, detergents, washes or buffers, blocking agents, mass spectrometry matrix-forming materials, antibody capture agents, positive control samples, negative control samples, software and information, such as protocols, guidance and reference data) for processing a biological sample.

In general, immunoassays involve contacting a sample containing or suspected of containing a biomarker of interest with at least one antibody that binds specifically to the biomarker. A signal is then generated indicating the presence or amount of complexes formed by polypeptides binding to antibody in the sample. The signal is then related to the presence or amount of the biomarker in the sample. Numerous methods and devices are well known to those skilled in the art for detection and analysis of biomarkers. See, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792 and the publication “The Immunoassay Handbook, by David Wild” (ed. Stockton Press, New York, 1994).

The devices and assay methods known in the prior art may utilize labeled molecules in various sandwich assay formats, competitive or non-competitive, to generate a signal that is related to the presence or amount of the biomarker of interest. Suitable assay formats also include chromatographic, mass spectrometry and protein blotting methods. In addition, certain methods and devices, such as biosensors and optical immunoassays, may be employed to determine the presence or amount of analytes without the need for a labeled molecule. See, for example, U.S. Pat. Nos. 5,631,171 and 5,955,377, each of which is incorporated herein by reference in its entirety, including all tables, figures and claims. A skilled person in the art also recognizes that robotic instrumentation including, but not limited to, the Beckman ACCESS®, Abbott AXSYM®, Roche ELECSYS®, Dade Behring STRATUS® systems are among the immunoassay analyzers that are capable of performing immunoassays. But any suitable immunoassay can be used, for example, enzyme-linked immunoassays (ELISA), radioimmunoassays (RIAs), competitive binding assays and similar ones. Such instruments can, in general, be designed to analyze all or part of a sample introduced into the instrument for the analyte(s) of interest, for example, by causing all or part of a sample to be contacted with a binding agent specific for each analyte of interest and detecting bound complexes of the analyte with its corresponding binding agent.

The antibodies or other polypeptides can be immobilized on a variety of solid supports for use in assays. The solid phases that can be used to immobilize specific binding members include those developed and/or used as solid phases in solid phase binding assays. Examples of suitable solid phases include membrane filters, cellulose-based papers, granules (including polymer, latex and paramagnetic particles), glass, silicon wafers, microparticles, nanoparticles, TentaGels, AgroGels, PEGA gels, SPOCC gels and multi-well plates. A test strip can be prepared by coating the antibody or a plurality of antibodies on a solid support matrix. This strip can then be dipped into the test sample and then quickly processed by means of washings and detection steps to generate a measurable signal, such as a colored spot. The antibodies or other polypeptides can be bound to specific zones of assay devices by direct conjugation to an assay device surface or by indirect binding. In an example of the latter case, antibodies or other polypeptides can be immobilized on particles or other solid supports, and that solid support immobilized on the device surface.

Biological assays require detection methods and one of the most common methods for quantifying results is to conjugate a detectable marker to a protein or nucleic acid that has affinity for one of the components of the biological system under study. Detectable markers can include molecules that are detectable (for example, fluorescent portions, electrochemical markers, metal chelates, etc.), as well as molecules that can be detected indirectly by producing a detectable reaction product (for example, enzymes such as horseradish peroxidase, alkaline phosphatase, etc.) or by a specific binding molecule that can be detectable (for example, biotin, digoxigenin, maltose, oligohistidine, 2,4-dinitrobenzene, phenylarsenate, ssDNA, dsDNA, etc.).

In certain aspects, the present invention provides kits for the analysis of the exertional rhabdomyolysis markers described herein. The kit comprises reagents for the analysis of at least one test sample comprising at least one antibody that is an exertional rhabdomyolysis marker. The kit may also include devices and instructions for performing one or more of the diagnostic and/or prognostic correlations described herein. Preferred kits will comprise a pair of antibodies for performing a sandwich assay, or a labeled species for performing a competitive assay, for the analyte. Preferably, a pair of antibodies comprises a first antibody conjugated to a solid phase and a second antibody conjugated to a detectable marker, wherein each of the first and second antibodies bind to a renal injury marker. More preferably, each of the antibodies are monoclonal antibodies. The instructions for using the kit and performing the correlations may be in the form of labeling, which refers to any written or recorded material that is attached or otherwise accompanies a kit at any time during its manufacture, transport, sale or use. For example, the term labeling encompasses advertising leaflets and brochures, packaging materials, instructions, audio or video cassettes, computer disks, as well as writings printed directly on the kits. transport, sale or use.

In one embodiment, the biological sample can be obtained from any individual, whether a healthy individual, exhibiting, suspected of having, or being treated for ER.

In one embodiment, the samples to be analyzed are prepared from urine collected from an individual.

In one embodiment, considering that the identification of these marker molecules can also be obtained from blood samples, the method can also be applied to blood samples, when they are analyzed by any assay methodology that is capable of measuring differences in the levels of the selected marker(s) in a sample.

In one embodiment, an individual undergoing strenuous physical exercise has urine and/or blood samples collected periodically, before, during and after the end of each physical exercise practice, to evaluate the propensity for developing rhabdomyolysis.

In one embodiment, the biomarker of a sample is at least 2.5 times higher compared to an expected level in a resting group. In one embodiment, the biomarker of a sample is at least 2.0 times higher compared to an expected level in a resting group. In one embodiment, the biomarker of a sample is at least 1.5 times higher compared to an expected level in a resting group. In one embodiment, the biomarker of a sample is at least 1.25 times higher compared to an expected level in a resting group. In one embodiment, the biomarker of a sample is at least 2.5 times lower compared to an expected level in a resting group.

In another embodiment, an individual may have blood and/or urine samples collected before, during or after strenuous physical exercise whether it may be and its purpose such as in a gym, military missions, a sports competition or work in agriculture, considering the particularities of each sports modality regarding the intensity, duration and possibility of collecting biological samples, to monitor their health and assess the possible development of rhabdomyolysis in real time.

In one embodiment, the application of the present diagnostic method is indicated for investigation of rhabdomyolysis in an individual with serum CK activity higher than 5 times the upper limit of normality (of 174 IU/L), associated or not with anamnestic report that indicates strenuous physical activities or subjective conditions associated with the understanding of the term “strenuous physical activity”.

In one embodiment, in the absence of the results of laboratory tests, such as, serum CK level, due to the severity of an individual condition, the medical team may choose to use the investigation with the marker panel to proceed and expedite the choice of therapeutic conducts.

In one embodiment, an individual in medical care presents myoglobinuria and also reports previous practice of strenuous physical exercises, such as sports, military or agricultural activities, being indicated the application of the present diagnostic method while the laboratory tests are being performed.

In one embodiment, an individual enters the hospital emergency room with acute myocardial infarction, preceded by report of strenuous physical activity, such as sports, military or agricultural activities, being indicated the application of the present diagnostic method while other tests are being performed.

In one embodiment, an individual enters the hospital emergency room with acute renal failure, preceded by report of strenuous physical activity, such as sports, military or agricultural activities, being indicated the application of the present diagnostic method while other tests are being performed.

In one embodiment, an individual enters the hospital emergency room with RE, preceded by report of strenuous physical activity and use of anabolic steroids and/or drugs of abuse, being indicated the application of the present diagnostic method while other tests are being performed.

In one embodiment, an individual enters the hospital emergency room with ER condition, preceded by report of consumption of possibly contaminated food, being indicated the application of the present diagnostic method while other tests are being performed.

In another embodiment, the present diagnostic method can be used in association with the investigation of other markers not belonging to the said panel of proteins, to elucidate the complementary diagnoses of associated conditions.

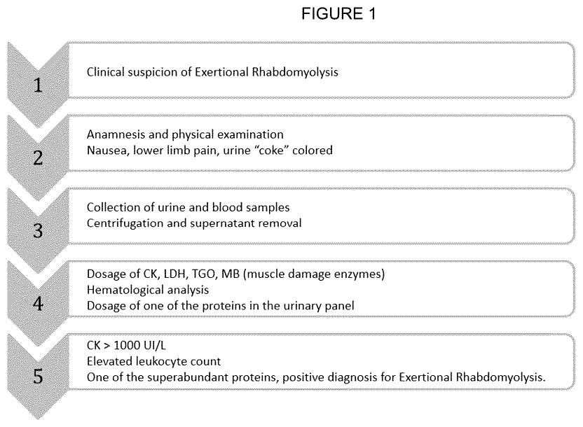

In one embodiment of the present method, schematically represented in FIG. 1, steps are illustrated in which:
(1) an individual is indicated with clinical suspicion of exertional rhabdomyolysis;
(2) an anamnesis of this individual is performed, observing particularly symptoms such as nausea, lower limb pain and dark urine, color of “coke”;
(3) urine and blood samples are collected from the individual for analysis, with the urine sample being centrifuged and the supernatant analyzed;
(4) it is carried out CK, LDH, AST and, optionally, myoglobin (MB) dosage, hematological analysis and dosage of two or more proteins of the biomarker panel in the urine sample;
(5) rhabdomyolysis is diagnosed if it is noted: elevated leukocyte count, CK less than 1000 IU/L and at least two of the proteins of the biomarker panel are differentially regulated, i.e., if they are overexpressed and underexpressed.

### EXAMPLES OF EMBODIMENT OF THE INVENTION

In what follows, exemplary embodiments of the object described herein are presented, in a non-restrictive manner, illustrating methods and results achieved by the same.

### Example 1: Identification of Differentially Regulated Proteins Related to Strenuous Exercise in Military Activity

This example describes the experimental design, the assays used to analyze samples and controls for the identification of the biomarkers, and presents statistical methods to confirm the significance of the results and their correlations with other biomarkers, used in the current technique to diagnose ER. The assays were performed on a group of individuals before practicing strenuous physical activity (M1) and after practicing strenuous physical activity (M2), from whom biological samples are withdrawn for monitoring progression of risk of ER through analysis of such biological samples, according to the present method. The steps for analyzing the samples include: sample withdrawal from the individual, sample filtration, protein breakdown (e.g., with addition of trypsin), separation of the markers (e.g., by chromatography), analysis of the markers (e.g., by mass spectrometry), and statistical analysis of the results.

### 1. Study Design and Exercise Training Program

A longitudinal/prospective study was conducted with Brazilian Navy soldiers enrolled in a nine-month special training program involving four phases. In the first phase (lasting 8 weeks), which consisted of a physical training, the clinical and demographic parameters of 31 soldiers, all men and asymptomatic, were measured.

The clinical and demographic parameters, defined based on this group, were: age (years): 29.0±2.7; total body mass (kg): 79.9±7.1; height (cm): 177.2±6.3; body mass index (kg.m−2): 25.3±1.8; Percentage of body fat (%): 10.4±2.9: Lean body mass (kg): 71.6±5.8; VO 2 max (mL.kg−1.min−1): 52.3±2.0.

The second and third phases consisted of training the soldiers with basic combat instructions and development of planning activities. The fourth phase aimed to practice the activities learned in the previous phases. This phase was executed over six missions performed in different environments of Brazil. Fourteen soldiers finalized this last phase, 10 of them being selected for this study.

The samples from the 10 individuals were collected at two distinct moments of phase four for each soldier, being at a moment before the individuals were subjected to strenuous physical activities (M1) and a moment after the individuals were subjected to strenuous physical activities (M2), which were defined, respectively, as moments of missions of low and high physical stress. All participants received and signed the informed consent form in accordance with Resolution 466, of Dec. 12, 2012, of the National Health Council (Guidelines and Standards for Conducting Experiments with Human Beings), under Substantiated Opinion of the Naval Hospital Marcílio Dias number 2.219.303.

### 2. Collection of Body Fluids

The collection of blood and urine samples was performed during moments of the missions of light physical effort (M1) and strenuous physical effort (M2), transported in cold thermal box (4° C.) till the place of analysis and then frozen and stored at −80° C. Three blood samples per participant were collected from the antecubital arm using a disposable 20-gauge needle equipped with a Vacutainer tube holder (Becton Dickinson, Franklin Lakes, NJ, United States) or similar. Two serum samples were collected (totaling 16 mL of serum) and kept in Vacutainer tubes containing SST-Gel and in Vacutainer tubes containing EDTA anticoagulant. The urine samples were collected in a sterilized plastic bottle, numbered and sealed.

### 3. Hematological and Biochemical Analyses

As a hematological biomarker, leukocytes were analyzed using a fluorescent flow cytometry equipment from Sysmex, model XT-2000i, or similar equipment. As biochemical biomarkers, creatine kinase (CK) and its isoform (CKMB), phosphorus (P), aspartate transaminase (AST) and lactate dehydrogenase (LDH) were analyzed, using a multianalyzer equipment for clinical analysis from Johnson & Johnson, model Vitros 4600 Chemical System (Ortho-Clinical Diagnostics, Johnson and Johnson, Rochester, NY, United States), or similar equipment.

### 4. Sample Preparation for Proteomic Analysis

Urine samples (10 mL) were centrifuged for 15 minutes at 2,000×g and 4° C. Then, they were filtered with sterile syringe filter (0.22 μM) and concentrated with Millipore Amicon Ultra-15 10K (Merck Millipore) according to the manufacturer's instructions. The retained material was collected and the filter was washed with 200 μL of 4 M urea solution, and the washing solution was mixed with the retained material to form a solution of retained materials. The amount of proteins in this solution of retained materials was quantified using a kit that performs a quantitative assay based on fluorescence, called Qubit Protein Assay Kit (Invitrogen). A volume corresponding to 100 μg was collected from each sample and triethylammonium bicarbonate 50 mM (final concentration) was added. The proteins were reduced with dithiothreitol (DTT) 10 mM for 40 minutes at 30° C. and alkylated with iodoacetamide (IAA) 40 mM for 30 minutes at room temperature in the dark. Before digestion, the samples were diluted to 0.8 M urea (final concentration) and digested with trypsin (Promega) (1:50 m/m) for 16 h at 30° C. After digestion, all reactions were acidified with trifluoroacetic acid at 1% (v/v) and the tryptic peptides were purified by chromatographic method with a solid phase extraction (SPE) cartridge containing hydrophilic-lipophilic balance polymer solvent and reverse phase for all components. The system used was the OASIS HLB-SPE (Waters). Then, the samples were dried and solubilized in 0.1% formic acid (FA).

### 5. Mass Spectrometry Analysis

The tryptic peptides (1 μg) were analyzed with an LTQ-Orbitrap Velos ETD (Thermo Fisher Scientific) along with Easy NanoLC II (Thermo Scientific). The peptides were separated on a reverse phase pre-column ReproSil-Pur C18-AQ C18 (4 cm×100 μm inner diameter, for 5 μm particles) and subsequently eluted on an analytical column of 20 cm×75 μm inner diameter for 3 μm particles, ReproSil-Pur C18-AQ, over 105 min, using a linear gradient of 2-30%, followed by 20 min of 30-45% of mobile phase B (100% ACN; 0.1% formic acid). To acquire the mass spectrometry (MS) data, the data-dependent acquisition (DDA) mode was used operated by the XCalibur software (ThermoFisher). Survey scans (350-1,500 m/z) were acquired on the Orbitrap system with a resolution of 60,000 at m/z. The selection of the 20 main precursors followed by fragmentation using the collision-induced dissociation (CID) method was employed using normalized collision energy of 35. The MS/MS settings included: minimum signal required of 5,000, isolation width of 2.00, activation Q of 0.250 and activation time of 10 ms. A technical duplicate for each sample was performed.

### 6. Data Processing

The tandem mass spectrum data were searched using the Andromeda search algorithm and MaxQuant v.1.5.5.135 (10.1038/nprot.2016.136). The H. sapiens Swiss-Prot database (Uniprot, 20,359 entries, revised) was downloaded on Sep. 20, 2020. The search parameters were set to allow two missed tryptic cleavages; the oxidation of methionine and the carbamidomethylation of cysteine were set as variable and fixed modifications, respectively. Other search criteria included: mass tolerance of 20 ppm (precursor ions) and 0.5 Da (fragment ions) and minimum peptide length of 7 amino acids. A false discovery rate (FDR) of 1% was applied at the peptide and protein level. The “match between run” option was enabled. Protein quantification was performed using label-free quantification (LFQ), integrating the area of the extracted ion chromatogram (XIC).

### 7. Statistical Analysis

All data sets were tested for normal distribution, in order to apply parametric tests. For the proteomic data, the protein expression data were processed using the Perseus computational platform v.1.6.14.0 (http://www.coxdocs.org/doku.php?id=perseus:start). The LFQ data were transformed into log 2, and filtered to remove contaminants. Differentially regulated proteins (overexpressed or underexpressed) between the moment of low physical stress (M1) and the moment of high physical stress (M2) were determined using paired t-test of LFQ values and correction for FDR (false discovery rate) based on Benjamini-Hochberg (FDR<0.05). The correlation between biochemical markers and regulated proteins was determined by applying the Spearman correlation test. The Graphpad Prism 9.01® was used to perform the statistical analysis of the test and was considered significant with p≤0.05.

### 8. Bioinformatics Analysis

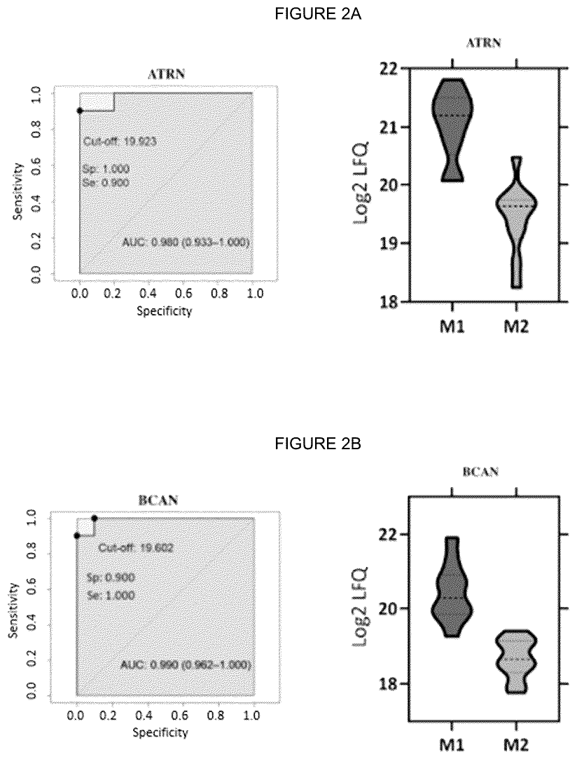

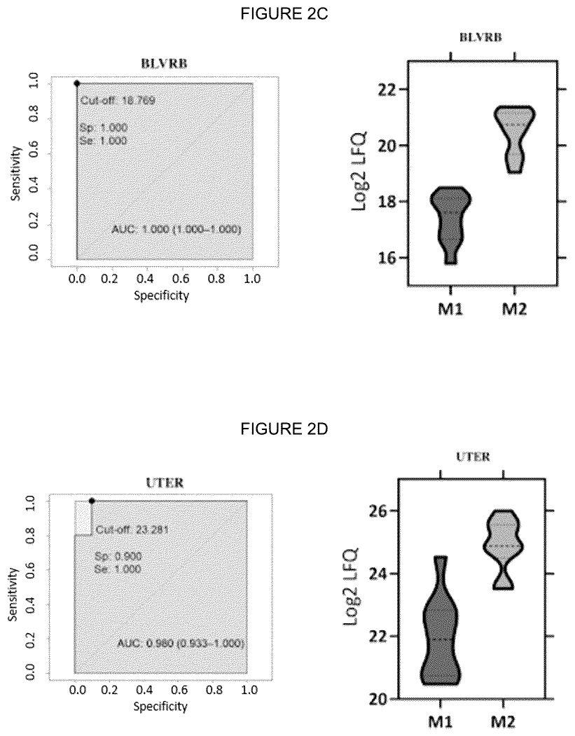

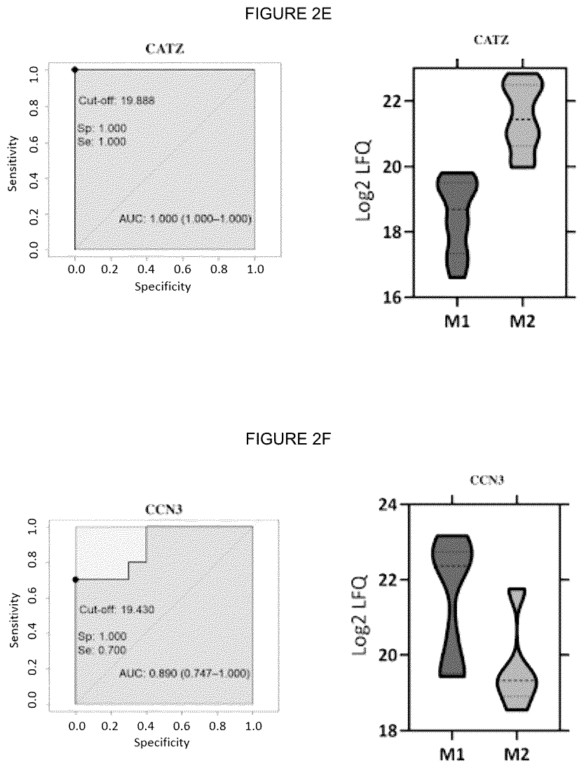

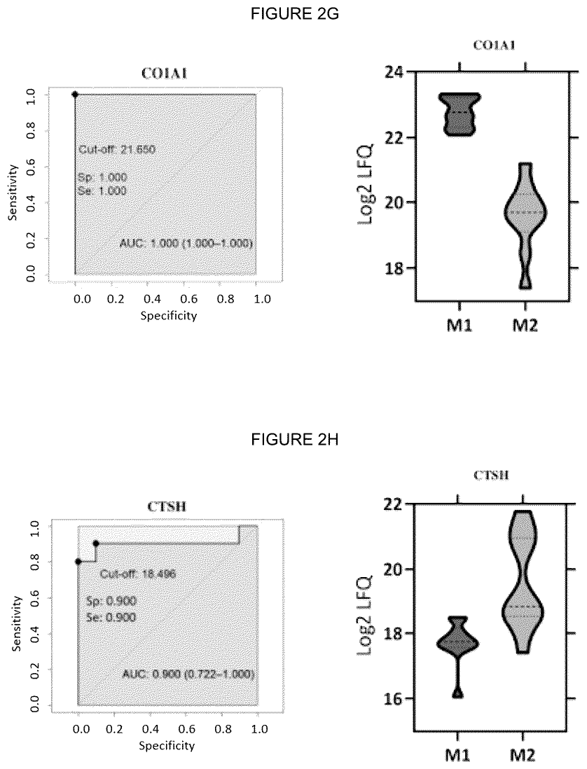

The ROC curves of FIGS. 2A to 2H, 3A to 3H and 4A to 4I were created using the pROC package (2011, BMC bioinformatics, 12, 77), available on the free platform Bioconductor. The complementary analyses were performed using common software of the technique, such as Perseus, Graphpad Prism v.9.01, RStudio software or similar.

### 9. Results

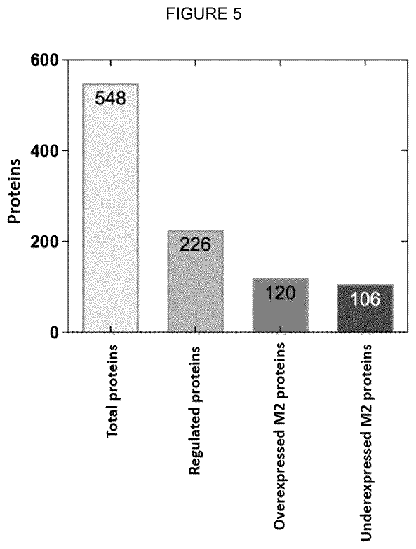

The LFQ approach (described in item 6 of this example) identified and quantified 548 urinary proteins. A total of 226 proteins were differentially regulated between the evaluated moments, being 120 overexpressed and 106 underexpressed, as observed in FIG. 5. A previous filter defined that only proteins with eight valid values in at least one group in both moments (M1, M2) are considered for the biomarker panel.

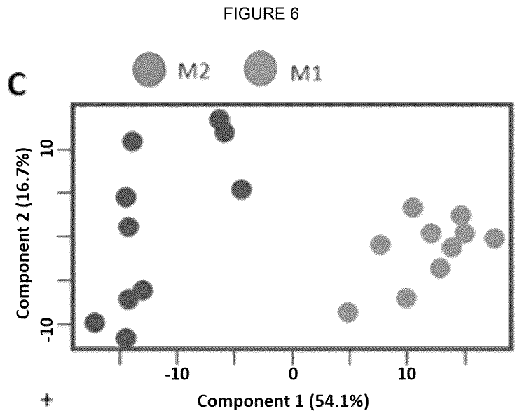

In order to identify a set of biomarkers associated with the occurrence of events, the combined set of control and initial event samples was verified using principal component analysis (PCA). The PCA displays the samples in relation to the axes defined by the strongest variations between all samples, without taking into account the outcome of the case or control, thus mitigating the risk of overfitting the distinction between case and control. As observed in FIG. 6, the multivariate PCA analysis showed a clear separation between the two groups evaluated, revealing that the proteomic profile differs according to the progression of physical exercise.

### Example 2: Correlation Between the Differentially Regulated Proteins After Physical Activity and the Classic Biochemical Markers of Rhabdomyolysis

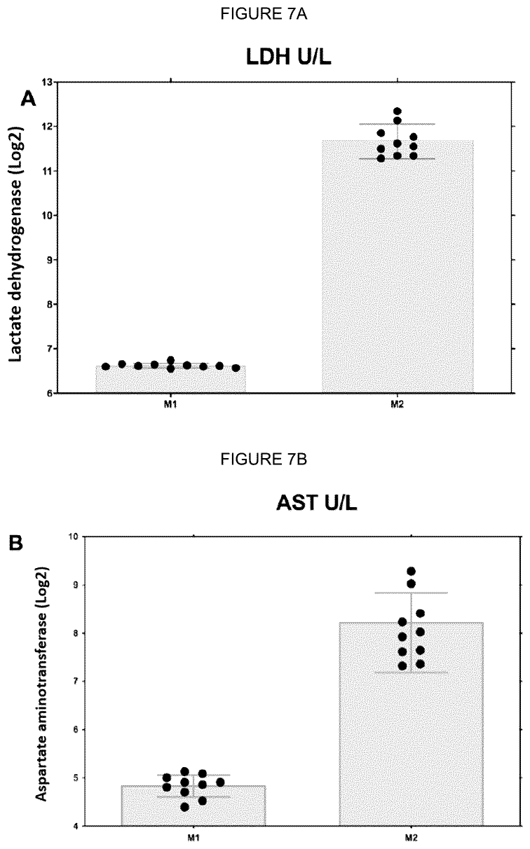

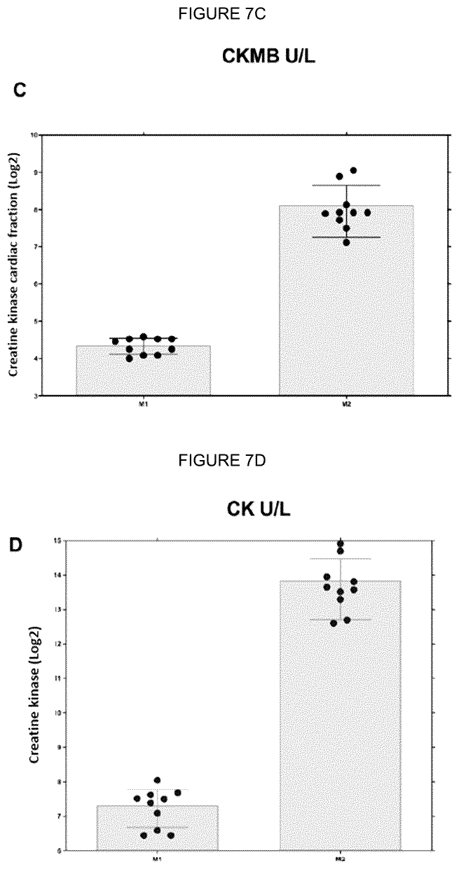

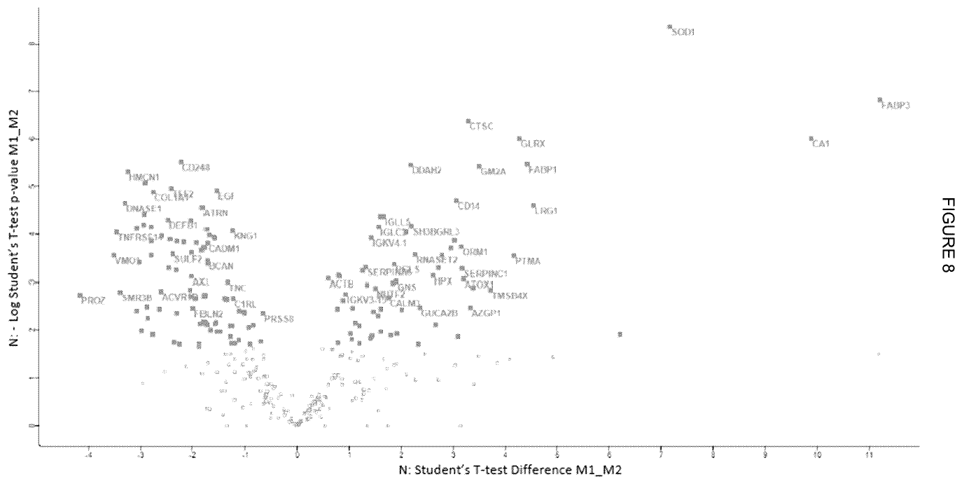

The quantification of the classic blood biochemical markers for rhabdomyolysis, LDH, AST, CKMB and CK, as well as the amount of total proteins in the urine show significantly higher levels at the time of intense physical activity (M2) when compared to the initial moment, before the intense physical activity (M1) (p-value<0.01), as illustrated in FIG. 7A to FIG. 7D.

Next, three of these (CK, LDH and AST), widely used in the clinic to assess the progression of rhabdomyolysis, were selected for correlation analysis to identify possible urinary protein biomarkers. As observed in Table 1, the proteins CTSH, PIK3IP1, DEFB1, ITGB1, BCAN, TNFRSF10C correlate simultaneously with the three main blood biomarkers of muscle damage used in clinical practice for the diagnosis of rhabdomyolysis (CK, LDH and AST). The proteins GLRX1, BLVRB, DSC2, CO1A1, S28A1, PGRP2, UTER and HBA have correlation with at least two blood biomarkers of muscle damage. The proteins GLRX1, BLVRB, DSC2, COL1A1, S28A1, GGCT, UTER, HBA, HMCN1, CCN3, PARK7, ICAM2, SLURP1, PGRP2, CATZ, FABP5, ATRN and PPIA have correlation with at least one blood biomarker of muscle damage.

Correlation analysis between the three biochemical markers traditionally used for the diagnosis of rhabdomyolysis and urinary protein abundance:

| | CK (U/L) | | LDH (U/L) | | AST (U/L) | |
|---|---|---|---|---|---|---|
| | spearman-r | valor-p | spearman-r | valor-p | Spearman-r | valor-p |
| CTSH*** 1 | 0.733 | 0.02023 | 0.648 | 0.04898 | 0.673 | 0.03897 |
| PIK3IP1 *** | −0.697 | 0.03056 | −0.782 | 0.01053 | −0.661 | 0.0438 |
| DEFB1*** | −0.673 | 0.03897 | −0.782 | 0.01053 | −0.661 | 0.0438 |
| ITGB1 *** | −0.661 | 0.0438 | −0.782 | 0.01053 | −0.721 | 0.02336 |
| BCAN *** | −0.661 | 0.0438 | −0.709 | 0.02675 | −0.709 | 0.02675 |
| TNFRSF10C | −0.661 | 0.0438 | −0.697 | 0.03056 | −0.648 | 0.04898 |
| *** | | | | | | |
| GLRX1** | −0.709 | 0.02675 | −0.6 | 0.07343 | −0.77 | 0.01256 |
| BLVRB** | −0.685 | 0.03465 | −0.552 | 0.10488 | −0.685 | 0.03465 |
| DSC2** | −0.648 | 0.04898 | −0.588 | 0.08061 | −0.661 | 0.0438 |
| CO1A1** | −0.636 | 0.05443 | −0.673 | 0.03897 | −0.721 | 0.02336 |
| S28A1** | −0.636 | 0.05443 | −0.745 | 0.01741 | −0.648 | 0.04898 |
| GGCT* 1 | 0.636 | 0.05443 | 0.685 | 0.03465 | 0.624 | 0.06032 |
| PGRP2** 1 | 0.576 | 0.08828 | 0.661 | 0.0438 | 0.661 | 0.0438 |
| UTER** | −0.539 | 0.11385 | −0.673 | 0.03897 | −0.697 | 0.03056 |
| HBA** 1 | 0.539 | 0.11385 | 0.648 | 0.04898 | 0.648 | 0.04898 |
| HMCN1* | −0.624 | 0.06032 | −0.733 | 0.02023 | −0.636 | 0.05443 |
| CCN3* | −0.6 | 0.07343 | −0.709 | 0.02675 | −0.612 | 0.06674 |
| PARK7* 1 | 0.6 | 0.07343 | 0.685 | 0.03465 | 0.564 | 0.09628 |
| ICAM2* | −0.588 | 0.08061 | −0.661 | 0.0438 | −0.576 | 0.08828 |
| SLURP1** | −0.576 | 0.08828 | −0.648 | 0.04898 | −0.455 | 0.19124 |
| CATZ* 1 | 0.564 | 0.09628 | 0.697 | 0.03056 | 0.624 | 0.06032 |
| FABP5* 1 | 0.564 | 0.09628 | 0.697 | 0.03056 | 0.588 | 0.08061 |
| ATRN* | −0.539 | 0.11385 | −0.673 | 0.03897 | −0.491 | 0.15482 |
| PPIA* 1 | 0.503 | 0.14403 | 0.661 | 0.0438 | 0.588 | 0.08061 |

A Spearman test was applied and the indices *, ** and *** indicate proteins that have correlation with one, two or three classic biochemical markers for rhabdomyolysis, respectively. Additionally, the overexpressed proteins are indicated with the index¹. The p-value for each correlation is presented.

Six proteins that showed simultaneous correlation with three classic biochemical markers (CTSH, PIK3IP1, DEFB1, ITGB1, BCAN, TNFRSF10C were submitted to ROC analysis (receiver operating characteristic), which considers the relative rates of true and false positives and negatives and generates an AUC curve (area under the ROC curve), whose area returns a value of 1.0 for a predictive capacity of 100%, and zero value when all predictions are wrong. All these proteins showed sensitivity and specificity above 80%.

Cathepsin H (CTSH), which belongs to the superfamily of cysteine proteases similar to papain and is involved in the degradation of intracellular proteins and extracellular matrix, since it is a lysosomal enzyme (2012, Journal of Neurochemistry, 122, 512-522). Other proteins of the cathepsin family such as CTSC and CTSK, have already been related to exercise, a previous study showed that in rats subjected to strenuous exercise, CTSC expression is increased (1984, Acta physiologica Scandinavica, 120 (1), 15-19). In addition, another study demonstrated that CTSK plays a key role in the loss of skeletal muscle and fibrosis, possibly through the reduction of inflammatory events (2016, PLOS One, 25;11(1), e0147198). The increase in CTSH abundance in conditions of strenuous exercise has not been previously described, being this the first report that we are aware of, as can be seen in FIG. 2H. Several evidences have shown a relationship between lysosomal cathepsins and renal pathology, one of the most lethal complications of rhabdomyolysis (2017, Frontiers in cell and developmental biology, 5, 114). Cathepsins B and D regulate extracellular matrix homeostasis, autophagy, apoptosis, glomerular permeability, endothelial function and inflammation (2017, Frontiers in cell and developmental biology, 5, 114).

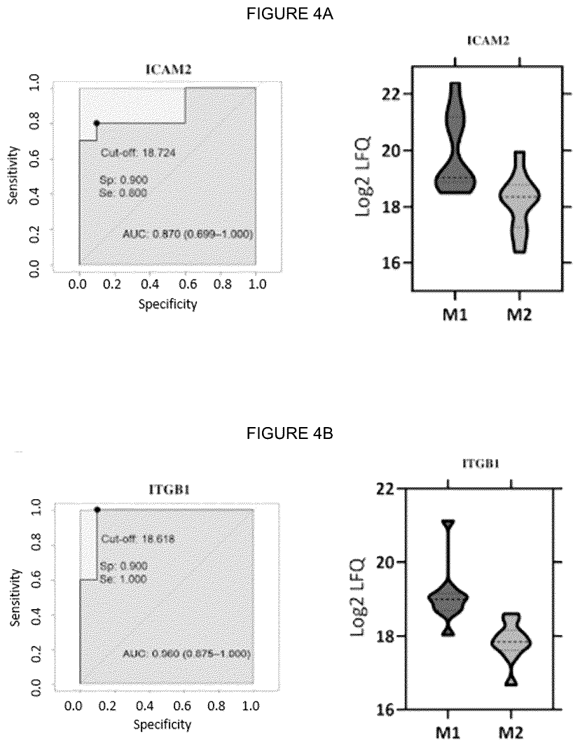

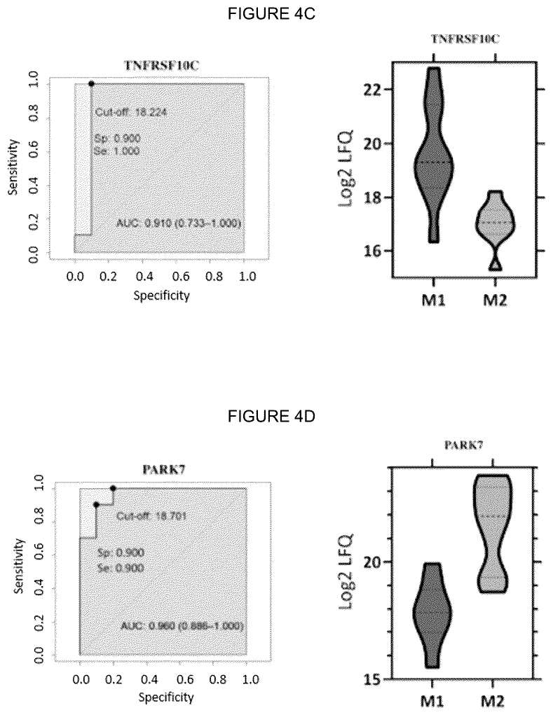

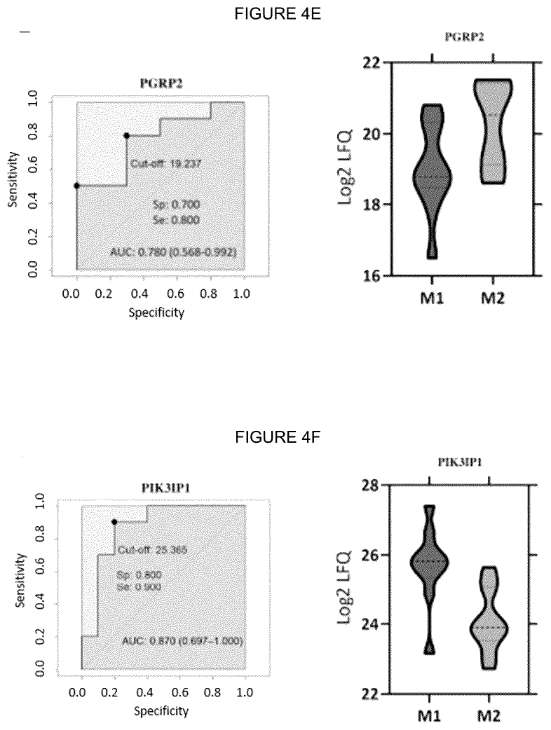

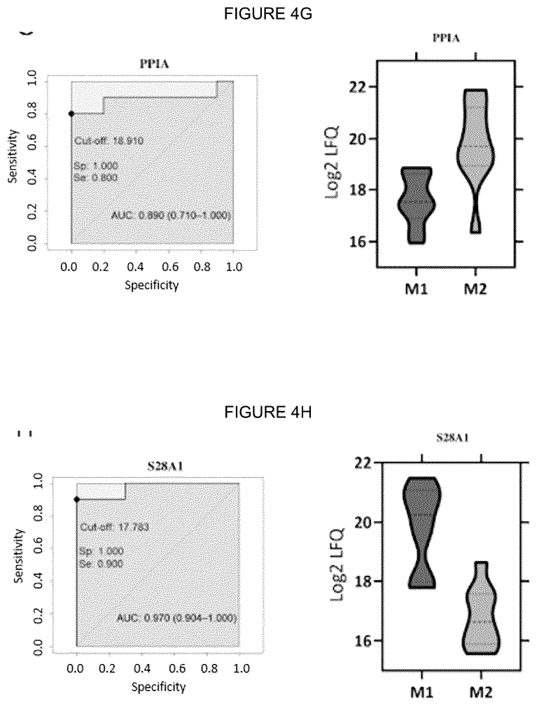

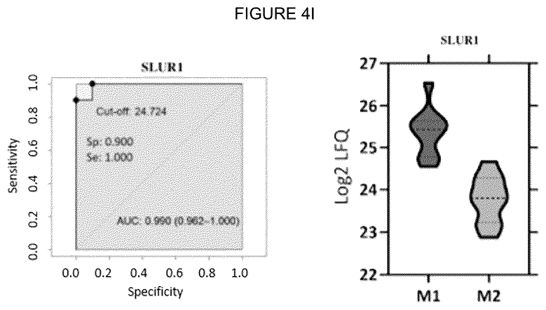

PIK3IP1 is a cell surface protein involved in the regulation of the PI3K pathway (2019, Clinical cancer research: an official journal of the American Association for Cancer Research, 25, 20). Herein, PIK3IP1 was identified as underexpressed in the group of samples after strenuous physical activity (M2), as FIG. 4F shows, and the abundance of this protein was correlated with muscle damage caused by strenuous physical activity. Recent evidence at the mRNA level indicated that PIK3IP1 is related to the regulator of the FOXO3 protein (2019, Laboratory investigation: a journal of technical methods and pathology, 99, 5). The inactivation of FOXO3, positive regulator of autophagy in skeletal muscle (1999, Cell, 96 (6), 857-868; 2006, Cell, 125 (5), 987-1001), showed to promote the downregulation of PIK3IP1 (10.1038/s41374-018-0184-7). In addition, a study already cited demonstrated that PIK3IP1 is an immunological regulator that inhibits the response of T cells (2019, Clinical cancer research: an official journal of the American Association for Cancer Research, 25, 20), which justifies the regulation of biological processes related to increased T cell activity in the group of samples after strenuous physical activity (M2) (T cell-mediated cytotoxicity and T cell-mediated immunity).

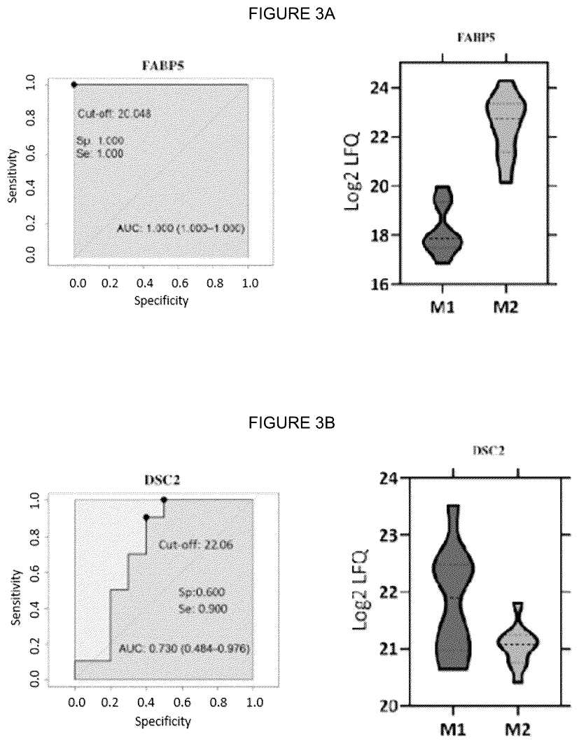

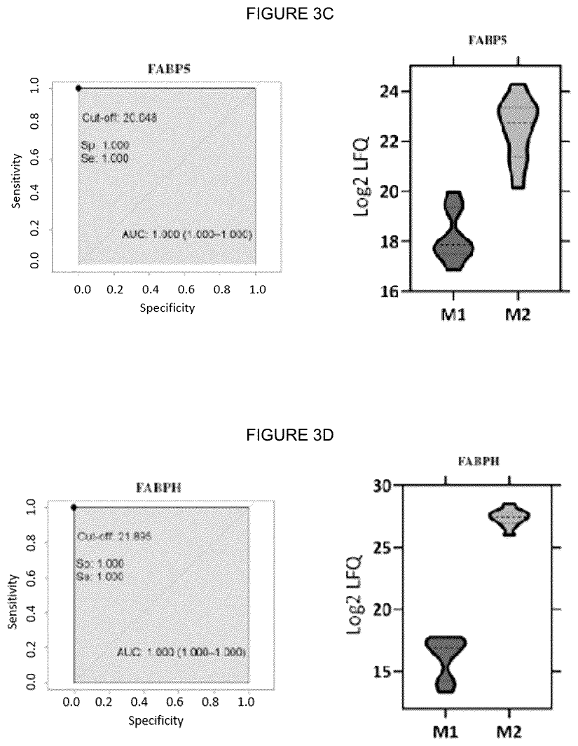

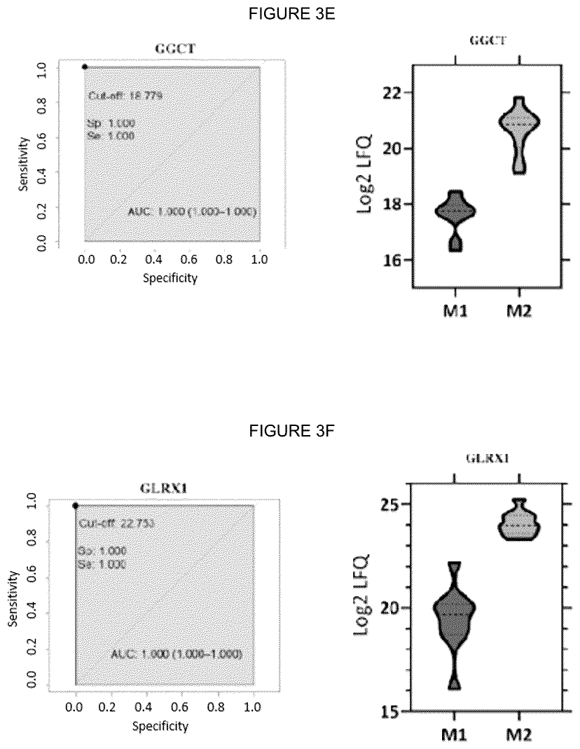

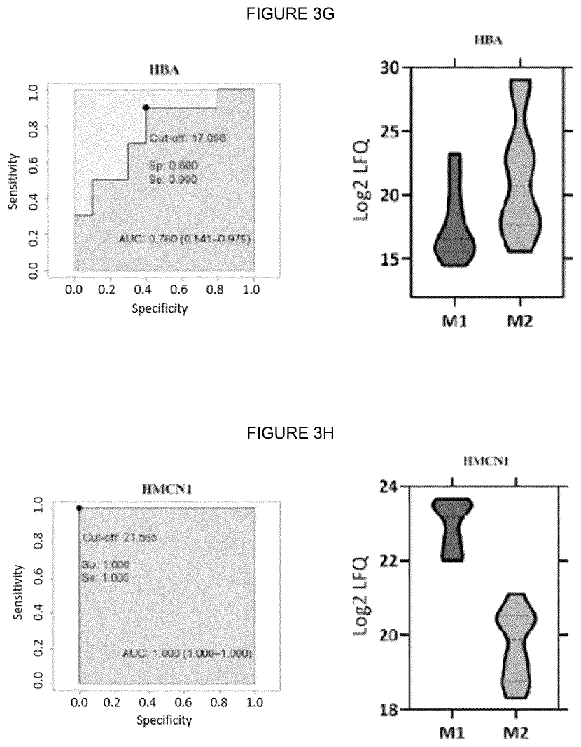

DEFB1 belongs to the family of defensins, which are antimicrobial and cytotoxic peptides (1994, Toxicology, 87 (1-3), 131-149). This family of peptides is associated with immune defense against bacteria, fungi and viruses (2015, Frontiers in immunology, 6, 115). Although the effects of inducing an immune response are well characterized, the involvement of the defensin family in anti-inflammatory activities has also been documented (2019, Seminars in cell & developmental biology, 88, 163-172). In the matter described here, DEFB1 was observed underexpressed in the group of samples after strenuous physical activity (M2), as FIG. 3A shows, when the participants accumulate more damage related to physical exercise. A document (2011, Journal of bioscience and bioengineering, 112 (2), 107-113) presented evidence of the relationship between DEFB1 and oxidative stress. The increase in DEFB1 levels was associated with the increase in the production of glutathione, an important tripeptide in protecting cells against oxidative stress. This finding indicates that there is an increase in processes related to oxidative stress after strenuous physical activity.

The ITGB1 protein is a member of the integrin family and is associated with cell de-adhesion. Here, this protein was identified as underexpressed after strenuous physical activity as shown in FIG. 4B. A study indicated that α7β1 integrin is related to the sensitization of skeletal muscle in response to mechanical stress and hypertrophy (2011, Journal of applied physiology (Bethesda, Md.: 1985), 111 (4), 1134-1141). Other studies have shown that α7β1 mRNA is positively regulated after eccentric contractions, probably as a source of resistance to injuries in repeated exercise sessions (2019, American journal of physiology. Cell physiology, 317 (4), C629-C641), suggesting that the increase in expression is related to muscle protection. Considering that the participants of the exemplary study presented herein were subjected to strenuous exercise conditions in M2, the data suggest that this protective effect was lost, since the levels of this protein were reduced.

The BCAN protein was identified as underexpressed in the group of samples after strenuous physical activity (M2), as shown in FIG. 2B. This protein is a neural proteoglycan that contributes to the formation of the extracellular matrix and is associated with various physiological processes and plasticity in the brain (2012, The international journal of biochemistry & cell biology, 44 (7), 1051-1054). A document demonstrated that intense exercise is capable of promoting cerebral mitochondrial dysfunction and thus disrupting the oxidative balance, resulting in the overproduction of free radicals (2008, Neurochemical research, 33, 51-58), indicating changes in the normal functions performed by the brain in the face of physical exercise. The matter herein revealed brings the first report of the underexpression of BCAN levels associated with physical activity. It is hypothesized that this urinary underexpression after strenuous physical activity would be a reflection of changes in brain physiological processes.

TNFRSF10C is an apoptosis-inducing protein often studied in the context of cancer (2018, PeerJ, 6, e5336). In addition, the tumor necrosis factor superfamily plays important roles in the activation, proliferation, differentiation and migration of immune cells to the central nervous system (2018, Science signaling, 11 (511), eaao4910). A document demonstrated that TNFRSF10C is a myokine, through mRNA sequencing of skeletal muscle to study the gene expression of secreted proteins in acute and long-term exercise (2017, Molecular metabolism, 6, 352-365) and corroborates the teachings of the present description, since at the protein level, TNFRSF10C is underexpressed at the time of high physical activity (M2), as shown in FIG. 4C.

Peptidoglycan recognition proteins (PGRPs or PGLYRPs) are innate immunity proteins that are conserved from insects to mammals, recognize bacterial peptidoglycan and function in antibacterial immunity and inflammation. Mammals have four PGRPs—PGLYRP1, PGLYRP2, PGLYRP3 and PGLYRP4. They are secreted proteins expressed in polymorphonuclear leukocytes (PGLYRP1), liver (PGLYRP2) or on body surfaces, mucous membranes and in secretions (saliva, sweat) (PGLYRP3 and PGLYRP4). All PGRPs recognize bacterial peptidoglycan (2014, Portuguese Society for Microbiology Magazine, 3, 1-7). Three PGRPs, PGLYRP1, PGLYRP3 and PGLYRP4 are directly bactericidal for Gram-positive and Gram-negative bacteria and have no enzymatic activity, while PGLYRP2 (FIG. 4E) is an N-acetylmuramoyl-L-alanine amidase that hydrolyzes the peptidoglycan of the bacterial cell wall. Peptidoglycan recognition proteins influence the pathogen-host interactions not only through their antibacterial or peptidoglycan-hydrolytic properties, but also through their pro-inflammatory and anti-inflammatory properties that are independent of their hydrolytic and antibacterial activities (2014, Janeway's Immunology, 8th edition, 97). PGRPs probably play a role in antibacterial defenses and in various inflammatory diseases. They modulate local inflammatory responses in tissues (such as arthritic joints) and there is evidence of association of PGRPs with inflammatory diseases, such as psoriasis.

Gamma-glutamyl cyclotransferase (GGCT) is an antioxidant defense enzyme directly involved in the metabolism of glutathione, an endogenous antioxidant. GGCT catalyzes the formation of 5-oxoproline from gamma-glutamyl dipeptides and may play a significant role in glutathione homeostasis. GGCT induces the release of cytochrome C from the mitochondria with the resulting induction of apoptosis (2006, Biochemical and biophysical research communications, 346, 454-460). In the present embodiment of the invention, it is observed that such marker was overexpressed in the samples examined after the practice of strenuous exercise (M2), as shown in FIG. 3E.

Proteomic studies have revealed the involvement of GGCT in glutathione metabolism, playing an important role in vascular, cardiac and cell survival function due to its antioxidant function (2005, Molecular immunology, 42, 987-1014; 2007, Journal of molecular and cellular cardiology, 43, 344-353). Glutathione deficiency is involved in the progression of cardiac remodeling in animal and human models. The findings on the involvement of differentially expressed proteins (overexpressed or underexpressed) linked to glutathione metabolism in this study suggest that the glutathione metabolism may be an interesting therapeutic target for the Rhabdomyolysis.

Collagen alpha1 (CO1A1) is a constituent protein of the extracellular matrix and controls the physiology of skeletal muscle through collagens, recently described as degraded by cathepsins. Type I collagen is a member of the group I collagen; Type I collagen is a heterotrimer consisting of 2 chains a1 (I) and one chain a2 (I), encoded, respectively, by the genes COL1A1 and COL1A2. Collagen is the most abundant extracellular matrix protein in humans and is the main structural protein of skin, bone, tendon, ligaments and cornea. In the present embodiment of the invention, it is observed that such a marker showed correlation with two classic markers of Rhabdomyolysis in samples examined before and after the practice of strenuous exercise (M2), as shown in FIG. 2G.

The heart fatty acid binding protein (FABPH or FABP3) was the protein with the highest relative abundance in samples after physical activity strenuous (M2), has a strong correlation with CATH (spearman-r=0.685 and p-value=0.035). Endurance physical training provides many adaptations of the skeletal muscle, including increased capacity of the oxidative metabolism of fatty acids and carbohydrates. An increase in fatty acid oxidation is facilitated by increased uptake of these molecules by myocytes (1997, Biochemical and biophysical research communications, 231, 463-465), their mitochondrial transport and subsequent B-oxidation (2002, American journal of physiology. Endocrinology and metabolism, 283, E66-E72).

Endurance training increases the capacity of fatty acid utilization. Since it is believed that fatty acids enter the cells through facilitated diffusion, a possible mechanism behind this adaptation to training may be an increase, induced by exercise, of putative fatty acid transporters in the content of the sarcolemma membrane (1997, Biochemical and Biophysical Research Communications, 231, 463-465).

The family of fatty acid binding proteins stood out in the analysis of the urinary proteome after strenuous exercise. Several fatty acid binding proteins were overexpressed in M2, among them FABP3, which presented the highest abundance in urine after exercise, as shown in FIG. 8. Some studies evidenced that FABP3 is a valuable blood biomarker for detection of muscle injury, as well as modulates the absorption of fatty acids in the cells muscles (skeletal muscle and heart) and in other cells (liver, brain, small intestine) (2008, Toxicological Sciences, 103, 382-396; 2015, Journal of neuromuscular diseases, 2(3), 241-255; 2016, Toxicological sciences, 150, 247-256; 2017, Toxicologic Pathology, 45, 943-951; 2017, International journal of rheumatic diseases, 20 (2), 252-260; 2019, Expert review of molecular diagnostics, 19 (8), 739-755). Another study showed that blood FABP3, along with other biomarkers, was predictive for skeletal muscle necrosis (2008, Toxicological Sciences, 103, 382-396). Thus, when blood FABP3, when used in conjunction with classic muscle injury biomarkers, such as CK and AST, improved the sensitivity and specificity of the diagnoses of skeletal muscle injury in mice (2016, Toxicological sciences, 150, 247-256). FABP3 showed a higher association with muscle weakness in patients with polymyositis and dermatomyositis than serum levels of CK and myoglobin (2017, International journal of rheumatic diseases, 20 (2), 252-260). A recent study showed that the serum levels of FABP3 may represent a new biomarker for Duchenne muscular dystrophy (2019, Expert review of molecular diagnostics, 19 (8), 739-755). So far, there is no knowledge of commercially available urinary biomarkers for skeletal muscle damage by intense physical activity, which correlate significantly with blood muscle damage biomarkers after strenuous physical activity.

Additional clinical and laboratory tests can be combined, without prejudice to the result(s) of the assay of the biomarkers of the present invention. These include other biomarkers related to exertional rhabdomyolysis such as CK, LDH and AST.

Other clinical indications that can be combined without prejudice to the result(s) of the assay of the biomarkers of the present invention, include demographic information (for example, weight, sex, age, race), medical history (for example, family history, sickle cell trait, pre-existing disease, such as muscular dystrophies, proteinuria, renal failure or sepsis, type of drug exposure, such as NSAIDs, cocaine, statins), environmental variables (for example, intensity of physical activity, ambient temperature).

Other measures of muscle damage that can be combined with the result(s) of the marker assay are described below and in Harrison's Principles of Internal Medicine, 17th Ed., McGraw Hill, New York, pages 1741-1830, and Current Medical Diagnosis & Treatment 2008, 47 m Ed, McGraw Hill, New York, pages 785-815, each of which is incorporated herein by reference in its entirety.

### Example 3: Acute Renal Failure Caused by ER, After Strenuous Physical Training Military

This example describes the application of the marker panel in monitoring the progression of acute kidney injury caused by exertional rhabdomyolysis, after strenuous military physical training.

A 24-year-old patient came to the hospital complaining of nausea and vomiting, reporting having performed military physical training 20 hours earlier. The urine samples for the comparative proteomic analyses in this study were collected from the patient at 4 different times. The first collection (D0) was made at the time of his enrollment in the Amphibious Commands specialization course at CIASC (Centro de Instrução Almirante Sylvio de Camargo da Marinha do Brasil, in Rio de Janeiro). Three days later, the students participated in 12 hours of strenuous military physical training, and student 3201 developed Rhabdomyolysis due to effort with Acute Renal Failure. The hospitalization occurred the next day. Two other samples were collected 72 hours after training (D5_6 h and D5_14 h). The fourth sample evaluated was collected on the patient's discharge date (D12).

At the time of admission, his laboratory results showed a high level of serum CK of 9,300 U/L and high levels of urea (U) and serum creatinine of 147 mg/dl and 5.7 mg/dl, respectively. The urine test showed significant myoglobinuria on D5. The patient started dialysis on the second day of hospitalization, stopped on D3 and returned on D5 and remained on dialysis throughout the course. His laboratory rates normalized during his hospitalization and he was discharged on day 12.

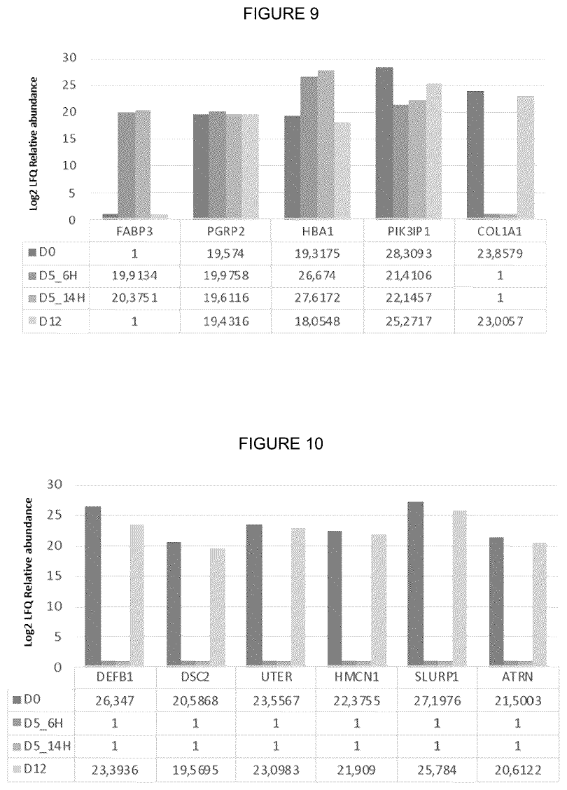

The analysis of the urinary proteome revealed the differentiation in the expression of three markers of the revealed panel, indicating that COL1A1 and DEFB1 were underexpressed and FABP3 was overexpressed during the evaluated hospitalization period, as shown in FIG. 9 (COL1A1 and FABP3) and FIG. 10 (DEFB1), compared to the quantitative results of these same proteins in the samples collected pre-training (D0) and after discharge (D12).

These data evidenced that the evaluation of urinary proteins from the markers revealed in this document is capable of monitoring the development and progression of Rhabdomyolysis.

Although exemplary modalities of the processes and products described have been presented in this report, it is not intended that the scope of protection be limited to the literalness of the same. Therefore, the description should be interpreted not as limiting, but merely as exemplifications of particular modalities that hold the inventive concept presented herein. A prior art technician will be able to readily apply teachings presented here in analogous solutions, resulting from the same, limited only by the scope of the claims of this request.

1. A method for proteomic investigation in a sample to diagnose rhabdomyolysis, comprising the steps of:
given a panel of biomarkers selected from the group consisting of: cathepsin H (CTSH); alpha-1 (I) collagen chain (CO1A1); phosphatidylinositol-3-kinase interaction protein 1 (PIK3IP1); beta-defensin 1 (DEFB1); integrin beta-1 (ITGB1); Brevican core protein (BCAN); a member of the tumor necrosis factor receptor superfamily 10C (TNFRSF10C); gamma-glutamylcyclotransferase (GGCT); Glutaredoxin (GLRX), Flavin reductase (NADPH) (BLVRB); Desmocollin-2 (DSC2); Alpha-1 (I) collagen chain (COL1A1); Sodium/nucleoside cotransporter 1 (S28A1); Uteroglobin (UTER); Alpha subunit of hemoglobin (HBA); Hemicentin-1 (HMCN1); CCN family member 3 (CCN3); Parkinson's disease protein 7 (PARK7); Intercellular adhesion molecule 2 (ICAM2); Secreted Ly-6/uPAR related protein 1 (SLURP1); N-acetylmuramoyl-L-alanine amidase (PGRP2); Cathepsin Z (CATZ); Fatty acid binding protein 5 (FABP5); Atractin (ATRN); Peptidyl-prolyl cis-trans isomerase A (PPIA) and heart fatty acid binding protein (FABP3);
detecting, in a biological sample withdrawn from an individual, at least two biomarkers from said panel that show significant differences in relative abundance compared to a set of reference values.

2. The method according to claim 1, further comprising obtaining the set of reference values from samples withdrawn from said individual before physical training.

3. The method according to claim 1, further comprising obtaining the set of reference values from samples withdrawn from a set of individuals before physical training.

4. The method according to claim 1, wherein the biological sample is a urine sample.

5. The method according to claim 1, wherein the biological sample is a blood sample.

6. The method according to claim 1, wherein the biological sample is incubated with an antibody or a protein antigen fragment that specifically binds to a protein or peptide fragment of a protein from said panel, under conditions that allow the specific antibody or antigen fragment to form a complex with:
(a) a peptide fragment of a protein from said panel; or
(b) the covalent or non-covalent complex of at least one molecule selected from the group comprising: peptide fragments of a protein from said panel; and, intact proteins from said panel, wherein the detection and measurement of the formed complex is made.

7. The method according to claim 6, wherein the detection and measurement of the formed complex is made by enzyme-linked immunoadsorption assay type ELISA.

8. The method according to claim 6, wherein the detection and measurement of the formed complex is made by mass spectrometry.

9. A proteomic investigation kit in a sample to diagnose rhabdomyolysis configured to perform the method of claim a, wherein the kit comprises:
a solid support that receives the biological sample;
a capture reagent, preferably an antibody, deposited on the solid support, to capture biomarkers from the panel present in the biological sample; and
means of detection and quantification of the captured panel biomarkers.

10. The kit according to claim 9, wherein the means of detection and quantification of the panel biomarkers in the biological sample are made of a material that emits fluorescence, chemiluminescence, turbidimetry, and nephelometry in contact with one of the panel biomarkers

11. The kit according to claim 10, wherein the means of detection and quantification of the panel biomarkers in the biological sample are constitute of a material that generates a signal in contact with one of the panel biomarkers and transmits the signal to a signal acquisition and data storage medium.
